(19) United States
(12) Patent Application Publication
(10) Pub. No.: US 2022/0105415 A1
(43) Pub. Date: Apr. 7, 2022

(54) TRAINING SUPPORT SYSTEM, TRAINING SUPPORT METHOD, AND TRAINING SUPPORT PROGRAM

(71) Applicant: TOYOTA JIDOSHA KABUSHIKI KAISHA, Toyota-shi Aichi-ken (JP)

(72) Inventors: Takuya Iwata, Toyota-shi Aichi-ken (JP); Issei Nakashima, Toyota-shi Aichi-ken (JP); Hisataka Yuasa, Nagoya-shi Aichi-ken (JP); Takeru Fukagawa, Chiryu-shi Aichi-ken (JP); Takashi Shimizu, Nisshin-shi Aichi-ken (JP); Yohei Otaka, Kariya-shi Aichi-ken (JP); Satoshi Hirano, Nagoya-shi Aichi-ken (JP); Masaki Katoh, Nagoya-shi Aichi-ken (JP); Yoko Inamoto, Nagoya-shi Aichi-ken (JP); Daisuke Imoto, Nagoya-shi Aichi-ken (JP)

(73) Assignee: TOYOTA JIDOSHA KABUSHIKI KAISHA, Toyota-shi Aichi-ken (JP)

(21) Appl. No.: 17/487,194

(22) Filed: Sep. 28, 2021

(30) Foreign Application Priority Data
Oct. 2, 2020 (JP) 2020-168108

(51) Int. Cl. A63B 71/06; G06F 16/24; G16H 70/60

(52) U.S. Cl. CPC A63B 71/0622; A63B 2214/00; G16H 70/60; G06F 16/24

(57) ABSTRACT

A training support system including an information acquisition unit configured to acquire trainee information including a symptom of a trainee, a database configured to store at least a plurality of symptoms and a plurality of training items in association with each other, an extraction unit configured to extract from the database one or more training items corresponding to the symptom of the trainee acquired by the information acquisition unit, and an output unit configured to output the one or more training items extracted from the database by the extraction unit.

### CROSS REFERENCE TO RELATED APPLICATIONS

This application is based upon and claims the benefit of priority from Japanese patent application No. 2020-168108, filed on Oct. 2, 2020, the disclosure of which is incorporated herein in its entirety by reference.

### BACKGROUND

The present disclosure provides a training support system, a training support method, and a training support program.

Japanese Patent No. 6052234 discloses a walking training apparatus for training a user to walk.

### SUMMARY

Recently, in addition to the walking training apparatus disclosed in Japanese Patent No. 6052234, various training systems have been developed for performing training for the purpose of functional recovery of a trainee's physical action. Under such a circumstances, it has become difficult for the trainee to select a training item suitable for him/her from among a large number of training items and then execute it.

The present disclosure has been made in view of the above circumstances, and an object of the present disclosure is to provide a training support system, a training support method, and a training support program which extract a training item suitable for recovery of a symptom of a trainee from among a plurality of training items and output it to thereby enable the trainee to perform effective training.

An example aspect of the present disclosure is a training support system including: an information acquisition unit configured to acquire trainee information including a symptom of a trainee; a database configured to store at least a plurality of symptoms and a plurality of training items in association with each other; an extraction unit configured to extract from the database one or more training items corresponding to the symptom of the trainee acquired by the information acquisition unit; and an output unit configured to output the one or more training items extracted from the database by the extraction unit. Since this training support system can output the training item(s) suitable for the recovery of the symptom of the trainee from among a large number of training items, the trainee can perform effective training by referring to the information.

The output unit is a display apparatus configured to display the one or more training items extracted from the database. In this way, the training item(s) suitable for the recovery of the symptom of the trainee become clearer.

In the database, the plurality of symptoms, a plurality of physical functions, a plurality of organs, and a plurality of training items are associated with each other, and in the database, the symptom of the trainee acquired by the information acquisition unit is selected from among the plurality of symptoms, one or more physical functions suspected to be impaired by the selected symptom is selected from among the plurality of physical functions, one or more organs related to the selected one or more physical functions are selected from among the plurality of organs, and one or more training items for the selected one or more organs are selected from among the plurality of training items, and the extraction unit is configured to extract the one or more training items selected in the database. In this way, the training support system can more accurately extract the training item(s) suitable for the recovery of the symptom of the trainee from among a large number of training items.

A priority may be set for each of the plurality of physical functions. When two or more of the training items are extracted from the database by the extraction unit, the output unit may be configured to output the two or more of the training items extracted from the database together with the priority set for each of the physical functions associated with the two or more of the training items. In this manner, since the trainee can recognize, for example, which training should be given priority among the plurality of training items presented, more effective training can be performed.

A priority may be set for each of the plurality of physical functions. When the two or more of the training items are extracted from the database by the extraction unit, the output unit may be configured to preferentially output the training item associated with the physical function having a higher priority rather than the training item associated with the physical function having a lower priority. In this manner, since the trainee can recognize, for example, which training should be given priority among the plurality of training items presented, more effective training can be performed.

The trainee information may further include a training item to be excluded, and the output unit may be configured not to output the training item to be excluded from among the one or more training items extracted from the database, or configured to output that the training item to be excluded is not a target of training. By doing so, it is possible to prevent the trainee from performing the training to be excluded by mistake.

The trainee information may further include information about a training time, and the output unit may be configured to output the training item which the trainee can perform within a training time from among the one or more training items extracted from the database. Since the training item which the trainee can perform within a limited training time is output, the trainee can perform effective training even within the limited training time.

Another example aspect of the present disclosure is a training support method including: acquiring trainee information including a symptom of a trainee; extracting one or more training items corresponding to the symptom of the trainee acquired in the acquiring from a database configured to store at least a plurality of symptoms and a plurality of training items in association with each other; and outputting the one or more training items extracted from the database. Since this training support method can output the training item(s) suitable for the recovery of the symptom of the trainee from among the large number of training items, the trainee can perform effective training by referring to the information.

Another example aspect of the present disclosure is a training support program causing a computer to execute: processing of acquiring trainee information including a symptom of a trainee; processing of extracting one or more training items corresponding to the symptom of the trainee acquired in the acquiring from a database configured to store at least a plurality of symptoms and a plurality of training items in association with each other; and processing of outputting the one or more training items extracted from the database. Since this training support program can output the training item(s) suitable for the recovery of the symptom of the trainee from among the large number of training items, the trainee can perform effective training by referring to the information.

According to the present disclosure, it is possible to provide a training support system, a training support method, and a training support program which extract a training item suitable for recovery of a symptom of a trainee from among a plurality of training items and output it to thereby enable the trainee to perform effective training.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing a configuration example of a training support system according to a first embodiment;

FIG. 2 is a flowchart showing an operation of the training support system shown in FIG. 1;

FIG. 3 shows an example of associations of symptom information, physical function information, organ information, and training items stored in a database;

FIG. 4 shows an example of a screen displayed on a display apparatus; and

FIG. 5 shows another example of a screen displayed on the display apparatus.

### DESCRIPTION OF EMBODIMENTS

Although the present disclosure is described below through the embodiments, the disclosure according to the claims is not limited to the following embodiments. In addition, not all of the configurations described in the embodiments are indispensable for solving the problems. For clarity of description, the following descriptions and drawings are been omitted and simplified as appropriate. In each drawing, the same elements are denoted by the same reference signs and repeated description is omitted as necessary.

### First Embodiment

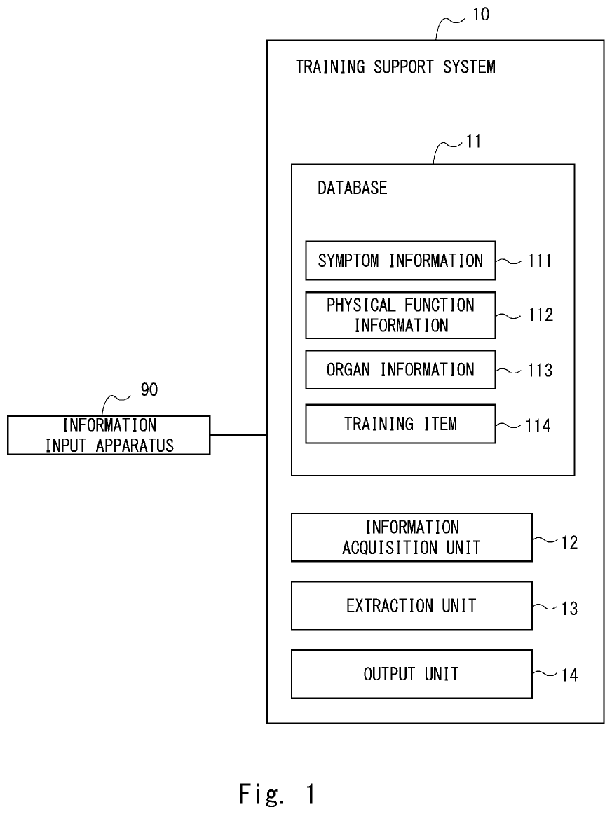

FIG. 1 is a block diagram showing a configuration example of a training support system 10 according to a first embodiment. In FIG. 1, in addition to the training support system 10, an information input apparatus 90 is also shown. The training support system 10 allows a trainee (a subject) to perform effective training by presenting training items suitable for recovery of the symptom of the trainee from among many training items.

Here, the trainees are those who suffer paralysis in a part of their bodies due to, for example, a stroke (cerebrovascular disorder) or spinal cord injury. The trainee's physical action functions deteriorates due to the above disease. In this embodiment, the physical action is an action included in, for example, Activities of Daily Living (ADL), and includes actions such as having a meal, going to the toilet, changing clothes, and walking.

The trainees need to perform several types of training to recover an impaired function of a physical action. Examples of the training in order to recover the functions for performing the physical action of having a meal include training of an eating action and a swallowing action. Examples of the training for recovering the function of performing a toilet action include training of a transfer action for transferring to a bed, a wheelchair, and a toilet seat, and training of an excretion activity in the bathroom. Examples of the training for recovering the function of a walking action include training for maintaining a sitting posture, training for maintaining a standing posture, training for walking on a flat ground, and training for walking on a slope. In addition to actively performing the physical actions as described above, the training may also include passive actions such as massaging, which involves receiving joint or muscle stimulation from a caregiver. In other words, there are a large number of types of training to be performed by the trainee. The training described above is also referred to as rehabilitation. For this reason, the training support system 10 can also be referred to as a rehabilitation support system.

The training support system 10 will be described in detail below.

As shown in FIG. 1, the training support system 10 includes a database 11, an information acquisition unit 12, an extraction unit 13, and an output unit 14.

The database 11 stores, for example, symptom information 111, physical function information 112, organ information 113, and training items 114 in association with each other.

The symptom information 111 includes a plurality of items of assumed symptoms. The plurality of items of the symptoms include, for example, “leakage from lips”, “poor bolus formation”, “poor delivery”, and “nasopharyngeal reflux”. Note that the items of the plurality of symptoms may be determined based on the symptoms of a plurality of other trainees who have been trained using the training support system 10 or the like in the past.

The physical function information 112 includes a plurality of items of physical functions suspected to be impaired by each symptom of the symptom information 111. The plurality of items of physical functions suspected to be impaired include, for example, items such as “oral cavity holding (function)” “mastication and bolus formation (function)”, “delivery into pharynx (function)”, and “nasopharyngeal closure (function)”. Note that the plurality of the items of physical functions may be determined based on the physical functions which have been confirmed to be impaired from the symptoms of the plurality of other trainees who have been trained using the training support system 10 or the like in the past.

The organ information 113 includes a plurality of items of organs related to each physical function of the physical function information 112. The items of the plurality of organs include, for example, items of “tongue”, “lips”, “lower jaw”, “cheek and palate”, and “soft palate”. Note that the items of the plurality of organs may be determined based on the organs related to the impaired physical functions of the plurality of other targets who have been trained using the training support system 10 or the like in the past.

The training item 114 includes a plurality of training items for each organ of the organ information 113. The plurality of training items include, for example, “tongue tip elevation exercise and tongue back elevation exercise”, “lip closure exercise”, “tongue twisting exercise, tongue tip elevation exercise, and tongue back elevation exercise”, “lower jaw up-and-down exercise”, “dexterity exercise”, “tongue back elevation exercise and tongue base retracting exercise”, and “blowing”. Note that the plurality of training items may be determined based on the training items for the organs related to the impaired physical functions of the plurality of other trainees who have been trained using the training support system 10 or the like in the past.

The information acquisition unit 12 is an interface for acquiring trainee information input to the information input apparatus 90 provided outside the training support system 10. The trainee information acquired by the information acquisition unit 12 includes at least information about the trainee.

The information input apparatus 90 is an apparatus for receiving information input by, for example, a character input operation using a keyboard, a mouse click operation, or a touch operation of a touch panel performed by the user. The information input apparatus 90 receives a predetermined operation performed by the user and outputs the trainee information set according to the received operation to the training support system 10. In the training support system 10, the information acquisition unit 12 acquires the trainee information output from the information input apparatus 90.

The information input apparatus 90 may be integrated with the training support system 10. Further, the information input apparatus 90 may be an apparatus including an information input/output function such as a smartphone or a tablet separate from the training support system 10, and may be connected to the training support system 10 so that communication is possible between them.

The extraction unit 13 extracts one or more training items corresponding to the symptom of the trainee acquired by the information acquisition unit 12 from the database 11. In the database 11, since the symptom information 111, the physical function information 112, the organ information 113, and the training item 114 are associated with each other as described above, when the symptom of the trainee acquired by the information acquisition unit 12 is input, one or more training items associated with this symptom are selected. The extraction unit 13 extracts one or more training items selected in the database 11.

The output unit 14 outputs one or more training items extracted from the database 11 by the extraction unit 13. In this embodiment, the case where the output unit 14 is a display apparatus for displaying one or more training items extracted from the database 11 by the extraction unit 13 will be described, but the present disclosure is not limited to this. The output unit 14 may be, for example, a speaker for outputting in audio one or more training items extracted from the database 11 by the extraction unit 13, or a transmission apparatus for transmitting one or more training items to a display apparatus provided outside the training support system 10.

### (Operation of Training Support System 10)

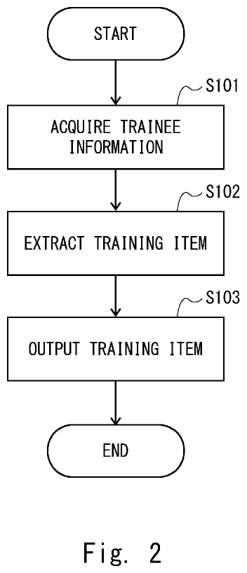

Next, an operation of the training support system 10 will be described with reference to FIG. 2.

FIG. 2 is a flowchart showing the operation of the training support system 10.

Note that it is assumed that the symptom information 111, the physical function information 112, the organ information 113, and the training item 114 are previously stored in the database 11 in association with each other.

First, the information acquisition unit 12 acquires the trainee information input to the information input apparatus 90 provided outside the training support system 10 (Step S101). The trainee information includes at least information about the symptom of the trainee.

After that, the extraction unit 13 extracts one or more training items corresponding to the symptom of the trainee acquired by the information acquisition unit 12 from the database 11 (Step S102).

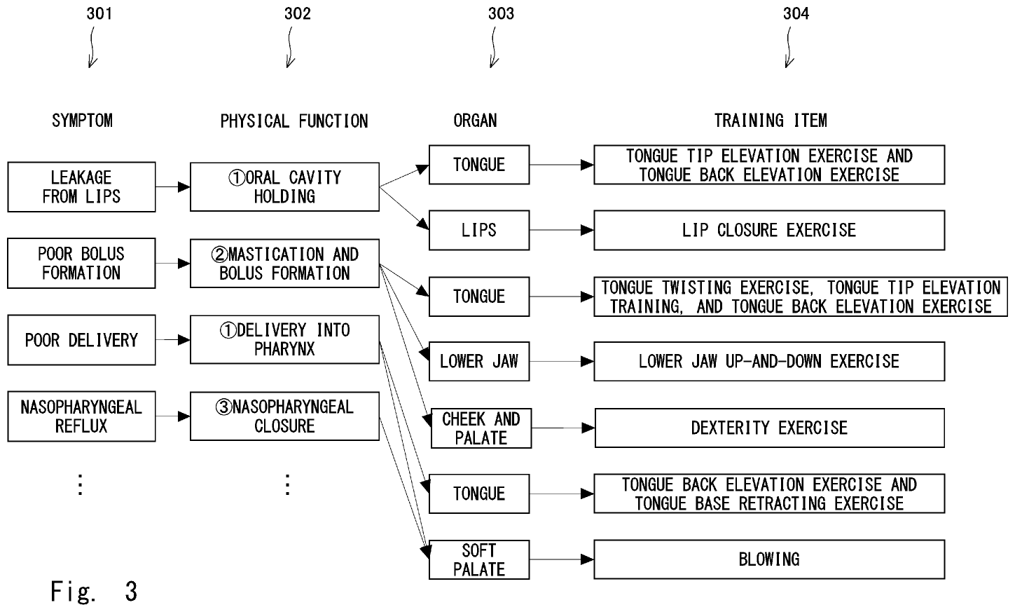

FIG. 3 shows an example of the association between the symptom information 111, the physical function information 112, the organ information 113, and the training item 114 stored in the database 11.

In the example of FIG. 3, “leakage from lips”, “poor bolus formation”, “poor delivery”, and “nasopharyngeal reflux” are included as an example 301 of items of the plurality of symptoms shown in the symptom information 111. Further, “oral cavity holding (function)”, “mastication and bolus formation (function)”, “delivery into pharynx (function)”, and “nasopharyngeal closure (function)” are shown as an example 302 of the plurality of items of physical functions suspected of impairment included in the physical function information 112. As an example 303 of a plurality of items of organs included in the organ information 113, “tongue”, “lips”, “lower jaw”, “cheek and palate”, and “soft palate” are shown. Further, as an example 304 of the plurality of training items included in the training item 114, “tongue tip elevation exercise and tongue back elevation exercise”, “lip closure exercise”, “tongue twisting exercise, tongue tip elevation exercise, and tongue back elevation exercise”, “lower jaw up-and-down exercise”, “dexterity exercise”, “tongue back elevation exercise and tongue base retracting exercise”, and “blowing” are shown.

In the example of FIG. 3, a priority is set for each item of the plurality of physical functions included in the physical function information 112. The priorities are set based on past performance, for example, the training items associated with higher priority physical functions are said to be effective in recovering the symptoms. In the example of FIG. 3, the highest priority “1” is set for the items of the physical functions “oral cavity holding” and “delivery into pharynx”, the next highest priority “2” is set for the item of the physical function “mastication and bolus formation”, and the lowest priority “3” is set for the item of the physical function “nasopharyngeal closure”.

Here, in the example of FIG. 3, the symptom of “leakage from lips” is associated with the function of “oral cavity holding” which is suspected to be impaired by this symptom. The function of the “oral cavity holding” is associated with the organs of the “tongue” and the “lips” related to this function. In addition, the organs of the “tongue” and the “lips” are respectively associated with the “tongue tip elevation exercise and tongue back elevation exercise” and the “lip closure exercise” which are the training items for these organs. Thus, in the database 11, when the “leakage from lips” is input as a symptom of the trainee, the “tongue tip elevation exercise and tongue back elevation exercise” and “lip closure exercise” finally associated with this symptom are selected as the training items.

In the example of FIG. 3, the symptom of “poor bolus formation” is associated with the function of “mastication and bolus formation” which is suspected to be impaired by this symptom. The function of the “mastication and bolus formation” is associated with the organs of the “tongue”, the “lower jaw”, and the “cheek and palate” related to this function. The organs of “tongue”, “lower jaw”, and “cheek and palate” are associated with the training items of “tongue twisting exercise, tongue tip elevation exercise, and tongue back elevation exercise”, “lower jaw up-and-down exercise” and “dexterity exercise”, respectively, which are the training items for these organs. Thus, in the database 11, when the “poor bolus formation” is input as the symptom of the trainee, the “tongue twisting exercise, tongue tip elevation exercise, and tongue back elevation exercise”, “lower jaw up-and-down exercise” and “dexterity exercise” finally associated with this symptom are selected as the training items.

In the example of FIG. 3, the symptom of “poor delivery” is associated with the function of “delivery into pharynx” which is suspected to be impaired by this symptom. The function of “delivery into pharynx” is associated with the organs of the “tongue” and the “soft palate” related to this function. The organs of the “tongue” and “soft palate” are associated with the training items of “tongue back elevation exercise and tongue base retracting exercise” and “blowing” which are the training items of these organs. Therefore, in the database 11, when the “poor delivery” is input as the symptom of the trainee, “tongue back elevation exercise and tongue base retracting exercise” and “blowing” finally associated with this symptom are selected as the training items.

Further, in the example of FIG. 3, the symptom of “nasopharyngeal reflux” is associated with the function of “nasopharyngeal closure” which is suspected to be impaired by this symptom. The function of “nasopharyngeal closure” is associated with the organ of the “soft palate” related to this function. The organ of “soft palate” is associated with “blowing” which is the training item for this organ. Therefore, in the database 11, when the symptom of “nasopharyngeal reflux” is input as the symptom of the trainee, the “blowing” finally associated with this symptom is selected as the training item.

The extraction unit 13 extracts one or more training items selected in the database 11. The example of FIG. 3 is merely an example, and it is needless to say that other symptoms, physical functions, organs, and training items may be further associated with the training item.

After that, the output unit 14 outputs one or more training items extracted from the database 11 by the extraction unit 13 (Step S103). In this embodiment, since the output unit 14 is a display apparatus, the output unit 14 displays one or more training items extracted by the extraction unit 13.

### (Example of the Display Screen)

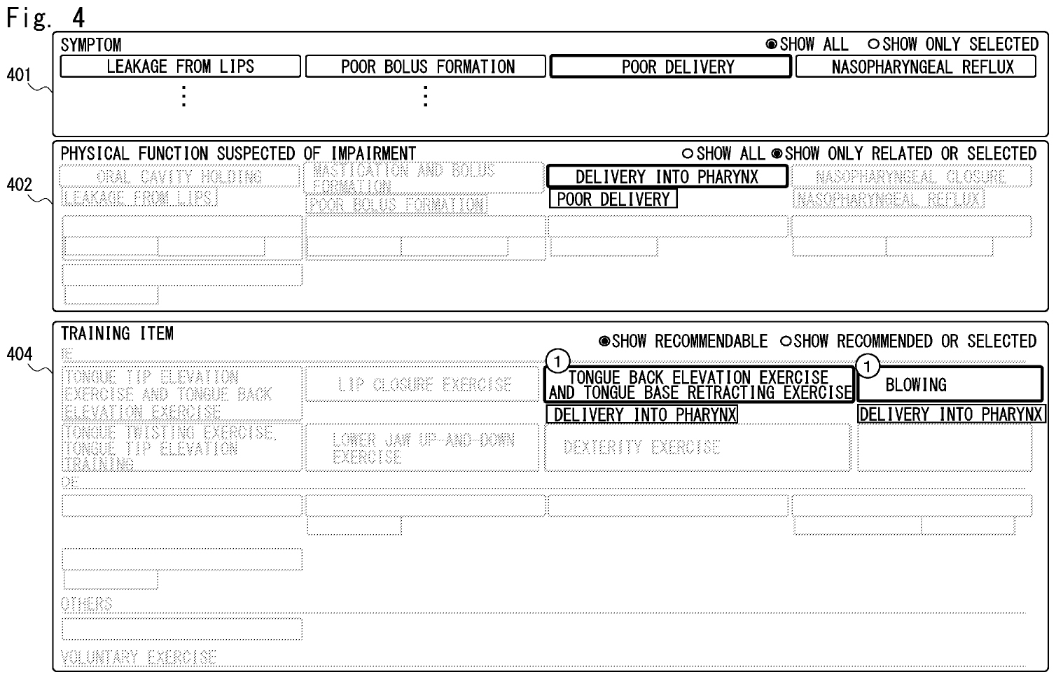

FIG. 4 shows an example of a screen displayed on the display apparatus (the output unit 14). In the example of FIG. 4, a plurality of items 401 of the symptoms, a plurality of items 402 of the physical functions suspected of impairment, and a plurality of items 404 of the training effective for recovery of the physical functions are displayed on a display screen.

In the example of FIG. 4, the “poor delivery” is selected as the symptom of the trainee. Thus, the items of the physical functions of the “delivery into pharynx” and the training items of the “tongue back elevation exercise and tongue base retracting exercise” and “blowing” associated with the symptom of the “poor delivery” are highlighted on the display screen of the display apparatus. Note that the training items of the “tongue back elevation exercise and tongue base retracting exercise” and “blowing” are associated with the item of the physical function “delivery into pharynx” for which the priority “1” is set. Thus, the training items of the “tongue back elevation exercise and tongue base retracting exercise” and “blowing” are displayed together with the priority “1”. In this way, the trainee or his/her caregiver can know the training item(s) suitable for the recovery of the symptom of the trainee from among the large number of training items, and the trainee can perform effective training without getting lost in the selection of the training items.

### (Another Example of Display Screen)

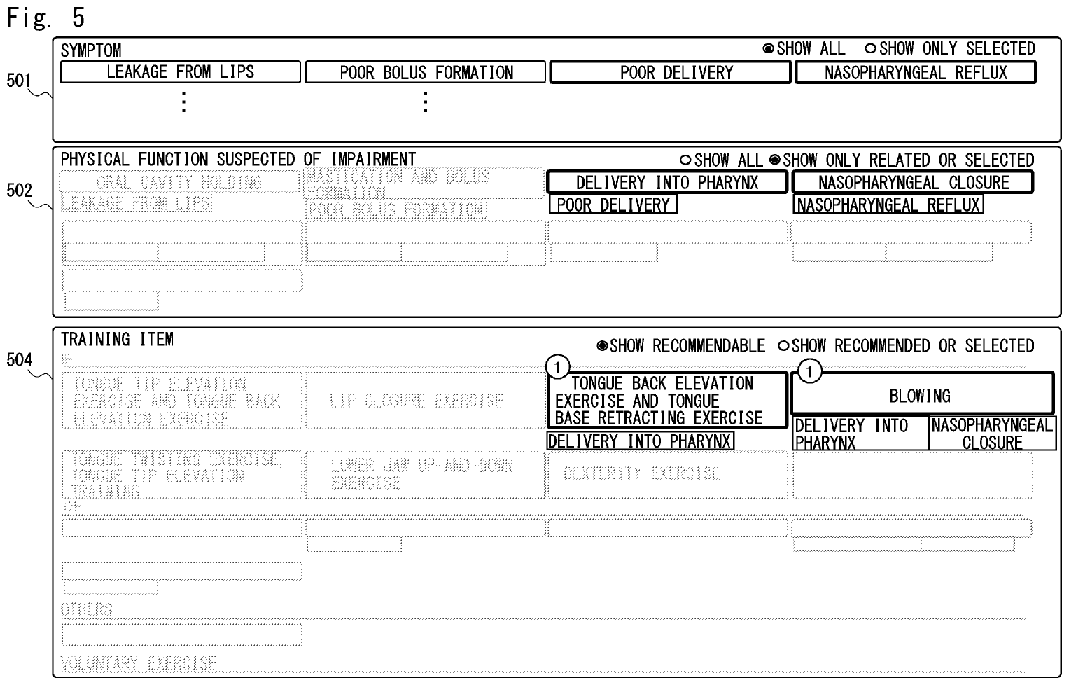

FIG. 5 shows another example of the screen displayed on the display apparatus (the output unit 14). In the example of FIG. 5, as in the example of FIG. 4, a plurality of items 501 of the symptoms, a plurality of items 502 of the physical functions suspected of impairment, and a plurality of items 504 of the training effective for recovery of the physical functions are displayed on the display screen.

In the example of FIG. 5, two items of “poor delivery” and “nasopharyngeal reflux” are selected as the symptoms of the trainee. Thus, the items of the physical functions of the “delivery into pharynx” and the training items of the “tongue back elevation exercise and tongue base retracting exercise” and “blowing” associated with the symptom of the “poor delivery” and “nasopharyngeal reflux” are highlighted on the display screen of the display apparatus. Note that the training items of the “tongue back elevation exercise and tongue base retracting exercise” and “blowing” are associated with the item of the physical function “delivery into pharynx” for which the priority “1” is set. Thus, the training items of the “tongue back elevation exercise and tongue base retracting exercise” and “blowing” are displayed together with the priority “1”. As in the example of FIG. 5, a plurality of the symptoms of the trainee may be selected (may be input).

As described above, since the training support system 10 according to this embodiment can output the training item(s) suitable for the recovery of the symptom of the trainee from among the large number of training items, the trainee can perform effective training by referring to the information.

In this embodiment, the case where the output unit 14, which is the display apparatus, displays one or more training items extracted by the extraction unit 13 together with the priority level has been described as an example, but the present disclosure is not limited to this. For example, the output unit 14, which is the display apparatus, may display one or more training items extracted by the extraction unit 13 in order of the priority from the upper side to the lower side of the screen.

In this embodiment, an example in which the training support system 10 extracts one or more training items corresponding to the symptom of the trainee from the database 11 and outputs the extracted training item(s) has been described, but the present disclosure is not limited to this. For example, the training support system 10 may be configured to extract one or more training items from the database 11 based on the symptom of the trainee, as well as at least one of the trainee's age, a gender, a harness used, and an assisting device used and then output the extracted training item(s). In this case, the trainee information acquired by the information acquisition unit 12 includes, in addition to the symptom of the trainee, at least any of the the trainee's age, the gender, the harness used, and the assisting device used. Also, in the database 11, for example, a combination of the symptom of the trainee and the trainee's age, the gender, the harness used, and the assisting device used, etc. is associated with the physical function information 112, the organ information 113, and the training item 114. In this way, the training support system 10 can more accurately extract the training item(s) suitable for the recovery of the symptom of the trainee and then output the extracted training item(s).

The trainee information acquired by the information acquisition unit 12 may further include a training item to be excluded. In this case, if one or more training items extracted from the database 11 by the extraction unit 13 include the training item to be excluded, the output unit 14 does not output the training item to be excluded. Alternatively, it is output that the training item to be excluded is not a target of training. Thus, it is possible to prevent the trainee from performing the training to be excluded by mistake.

Further, the trainee information acquired by the information acquisition unit 12 may further include information about training time. In this case, the output unit 14 outputs a training item which the trainee can perform within a training time from among the one or more training items extracted from the database 11 by the extraction unit 13. Since the training item which the trainee can perform within a limited training time is output, the trainee can perform effective training even within the limited training time.

Further, although the present disclosure has been described as a hardware configuration in the above embodiments, the present disclosure is not limited to this. The present disclosure can be realized by causing a CPU (Central Processing Unit) to execute a computer program for control processing by the training support system 10.

The above program includes instructions (or software codes) that, when loaded into a computer, cause the computer to perform one or more of the functions described in the embodiments. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A training support system comprising:
an information acquisition unit configured to acquire trainee information including a symptom of a trainee;
a database configured to store at least a plurality of symptoms and a plurality of training items in association with each other;
an extraction unit configured to extract from the database one or more training items corresponding to the symptom of the trainee acquired by the information acquisition unit; and
an output unit configured to output the one or more training items extracted from the database by the extraction unit.

2. The training support system according to claim 1, wherein
the output unit is a display apparatus configured to display the one or more training items extracted from the database.

3. The training support system according to claim 1, wherein
in the database, the plurality of symptoms, a plurality of physical functions, a plurality of organs, and a plurality of training items are associated with each other, and
in the database, the symptom of the trainee acquired by the information acquisition unit is selected from among the plurality of symptoms, one or more physical functions suspected to be impaired by the selected symptom is selected from among the plurality of physical functions, one or more organs related to the selected one or more physical functions are selected from among the plurality of organs, and one or more training items for the selected one or more organs are selected from among the plurality of training items, and
the extraction unit is configured to extract the one or more training items selected in the database.

4. The training support system according to claim 3, wherein
a priority is set for each of the plurality of physical functions,
when two or more of the training items are extracted from the database by the extraction unit, the output unit is configured to output the two or more of the training items extracted from the database together with the priority set for each of the physical functions associated with the two or more of the training items.

5. The training support system according to claim 3, wherein
a priority is set for each of the plurality of physical functions;
when the two or more of the training items are extracted from the database by the extraction unit, the output unit is configured to preferentially output the training item associated with the physical function having a higher priority rather than the training item associated with the physical function having a lower priority.

6. The training support system according to claim 1, wherein
the trainee information further includes a training item to be excluded, and
the output unit is configured not to output the training item to be excluded from among the one or more training items extracted from the database, or configured to output that the training item to be excluded is not a target of training.

7. The training support system according to claim 1, wherein
the trainee information further includes information about a training time, and
the output unit is configured to output the training item which the trainee can perform within a training time from among the one or more training items extracted from the database.

8. A training support method comprising:
acquiring trainee information including a symptom of a trainee;
extracting one or more training items corresponding to the symptom of the trainee acquired in the acquiring from a database configured to store at least a plurality of symptoms and a plurality of training items in association with each other; and
outputting the one or more training items extracted from the database.

9. A non-transitory computer readable medium storing a training support program causing a computer to execute:
processing of acquiring trainee information including a symptom of a trainee;
processing of extracting one or more training items corresponding to the symptom of the trainee acquired in the acquiring from a database configured to store at least a plurality of symptoms and a plurality of training items in association with each other; and
processing of outputting the one or more training items extracted from the database.
